(19) United States
(12) Patent Application Publication
(10) Pub. No.: US 2026/0315692 A1
(43) Pub. Date: Oct. 15, 2026

(54) MULTI-PUMP CLOSED-LOOP MANAGEMENT SYSTEM

(71) Applicant: CAREFUSION 303, INC., San Diego, CA (US)

(72) Inventors: Daniel M. ABAL, San Diego, CA (US); Brendan BURGESS, Poway, CA (US)

(21) Appl. No.: 19/104,182

(22) PCT Filed: Aug. 16, 2023
(86) PCT No.: PCT/US2023/030394

§ 371 Date: Feb. 14, 2025

Related U.S. Application Data
(60) Provisional application No. 63/398,819, filed on Aug. 17, 2022.

(51) Int. Cl. A61M 5/168; A61B 5/00; A61B 5/369

(52) U.S. Cl. CPC A61M 5/16804; A61B 5/369; A61B 5/4836; A61M 5/16863

(57) ABSTRACT

An infusion device control system intercepts a first command provided to a first infusion pump by a closed-loop control algorithm, the first command configured to adjust delivery of a first fluid by the first infusion pump according to sensor measurements provided to the closed-loop control algorithm. The control system receives a therapy target control parameter for modifying the delivery of the first fluid based on a real time parameter for delivering a second fluid by a second infusion pump, receives real time operating parameters and infusion status information associated with the first and second infusion pumps, and modifies the intercepted first command based on the real time operating parameters and infusion status information and the therapy target control parameter to control an effect of the deliveries of the first and second fluids, without providing input for modifying the intercepted first command to the closed-loop control algorithm.

### TECHNICAL FIELD

The present disclosure is generally related to managing infusions using closed-loop algorithms.

### BACKGROUND

Closed-loop control of intravenous (IV) infusions requires specific set up conditions and execute with specific tasks of controlling a single parameter according to a predetermined algorithm. Moreover, control-loop algorithms are optimized for specific IV infusion sets and devices. Modification of the closed-loop algorithms to adapt to different therapies, different sensors, or to control different types of infusion devices is costly and time consuming.

### SUMMARY

According to various aspects, the subject technology provides a closed-loop management system and method for controlling multiple infusion pumps. According to various aspects, a method includes intercepting a first command provided to a first infusion pump by a closed-loop control algorithm, the first command configured to adjust delivery of a first fluid by the first infusion pump according to sensor measurements provided to the closed-loop control algorithm; receiving a therapy target control parameter for modifying the delivery of the first fluid based on a real time parameter for delivering a second fluid by a second infusion pump; receiving real time operating parameters and infusion status information associated with the first and second infusion pumps; and modifying the intercepted first command based on the real time operating parameters and infusion status information and the therapy target control parameter to control an effect of the deliveries of the first and second fluids, without providing input for modifying the intercepted first command to the closed-loop control algorithm. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2A is a conceptual diagram illustrating an example system architecture, including an example resource management unit, according to aspects of the subject technology.

FIG. 2B illustrates the example system architecture of FIG. 2A with multiple infusion sets providing medications to a single extension set, according to aspects of the subject technology.

FIGS. 3A through 3C are first, second, and third example conceptual diagrams for managing multiple infusion pumps using the intelligent control module in a closed-loop or semi-closed-loop environment, according to various aspects of the subject technology.

FIG. 4 depicts an example process for managing multiple infusion pumps using an intelligent control system in a closed-loop or semi-closed-loop environment, according to various aspects of the subject technology.

FIG. 5 is a conceptual diagram illustrating an example electronic system for managing multiple infusion pumps using an intelligent control system in a closed-loop or semi-closed-loop environment, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

A portable infusion device control system is described herein that can connect to multiple infusion devices and control the infusion devices by repurposing existing closed-loop control algorithms. For example, a closed-loop algorithm may receive feedback information from the infusion device in the form of infusion information, and from the patient in the form of biometric and physiological sensor information. Based on this information, the algorithm may continuously control a physiological parameter based on manipulating control of the infusion device to dynamically adjust delivery of a medication to the patient according to a target parameter. The control system of the subject technology is configured to be positioned between the control-loop algorithm and the pump and, based on the same inputs- and potentially additional inputs intercept and modify pump commands sent by the control-loop algorithm to control the pump in accordance with real-time procedural needs in the field.

The subject technology provides a means to dynamically adapt closed-loop control algorithms for infusion devices without the effort of recoding or retraining the algorithm. The infusion device control system intercepts a command provided to a first infusion pump by a closed-loop control algorithm and receives a therapy target control parameter for modifying a delivery of the fluid. The system modifies the intercepted command based on real time operating parameters and infusion status information and the therapy target control parameter to control therapies provided by the infusion device, without providing input for modifying the intercepted command to the closed-loop control algorithm. In this way the closed-loop algorithm remains agnostic of the therapy and the pump.

The infusion device control system is further configured to repurpose and/or adapt an existing control-loop algorithm configured for one infusion device to control multiple infusion devices based on feedback information provided by the pump and associated sensors. In this regard, the patient sensor information is also provided to the control system, along with infusion information from the controlled pumps. The clinician selects the therapy and the number of pumps used in the therapy (which may be based on the selection) and the control system converts control parameters for a single therapy into control parameters for multiple therapies.

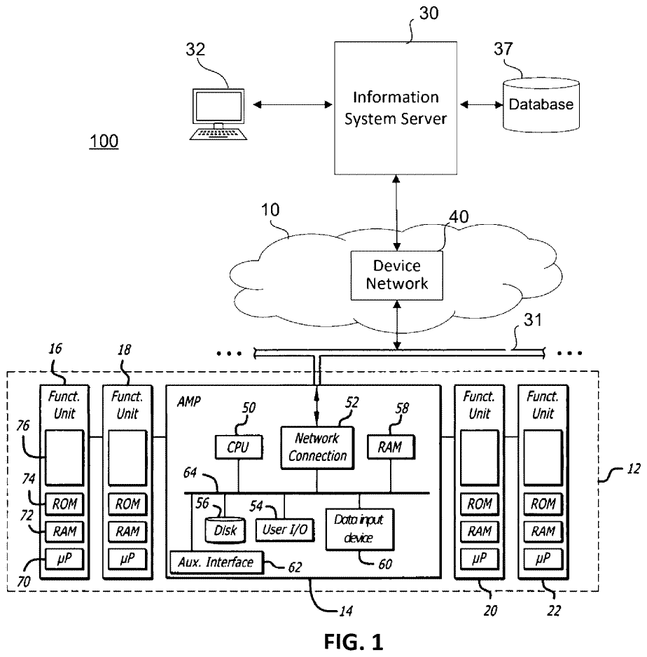

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 may be a wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistants, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a interface device 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include means (specifically configured with one or more features described) for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be a specifically configured device for entering coded data into a computer, such as a device(s) for reading a magnetic strip, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or other appropriate communication means. Auxiliary interface 62 may be an RS-232 communications interface, however other means for communicating with a peripheral device in the described environments such as a printer, patient monitor, infusion pump or other medical device may be used without departing from the subject technology. Additionally, data input device 60 may be a separate functional module, such as modules 16, 18, 20 and 22, and configured to communicate with controller 14, or other systems on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem, or a cable modem. Direct or indirect network connection may be used, including, but not limited to a telephone modem, an MIB system, an RS232 interface, an auxiliary interface, an optical link, an infrared link, a radio frequency link, a microwave link, a personal area network connection, a local area network connection, a cellular link, or a WLANS connection or other wireless connection.

Functional modules 16, 18, 20, 22 are specially configured devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be a patient treatment or monitoring device including, but not limited to, an infusion pump, a syringe pump, a patient-controlled analgesia (PCA) pump, an epidural pump, an enteral pump, a blood pressure monitor, a pulse oximeter, an EKG monitor, an EEG monitor, a heart rate monitor, or an intracranial pressure monitor or the like. Functional module 18, 20 and/or 22 may be a printer, scanner, bar code reader or other peripheral input, output, or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include specifically configured components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics, or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction, or a combination of both automated and manual interaction. Automated interaction may be continuous or intermittent and may occur through direct network connection 54 (as shown in FIG. 1), or through RS232 links, MIB systems, RF links such as BLUETOOTH, IR links, PANS, LANS, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication In some implementations, medication delivery modules 16, 18, 20, 22 include plug-in ports for expansion. Accordingly, a new medication delivery module may be attached to PCU 12 by coupling a connector through the plug-in ports, which may include electrical terminals so that the added medication delivery module 16, 18, 20, 22 may transmit and receive information to and from a control module 14. In some implementations, the added medication delivery module 16, 18, 20, 22 may also receive power from control module 14 through a plug-in port. Control module 14 may include a main display, a memory and a processor (see FIG. 10), and may be configured to display operational parameters and medication delivery status, and further information associated with each of medication delivery modules 16, 18, 20, 22. According to various implementations, module displays may also display physiological data (e.g., vital signs) associated with a patient.

A main display (e.g., I/O 54) may be configured to display one or more user interfaces for the display of operational parameters or other data associated with a module 16, 18, 20, 22, and/or physiological properties associated with the patient. The main display may include multiple user interfaces, with each individual user interface graphically displaying information for a respective one of medication modules, including information also displayed on a corresponding module displays. In some implementations, control module 14 includes a communications module (including, e.g., an antenna), configured to communicate wirelessly with a controller, or with a network.

With reference to FIG. 1, when a medication delivery module 16, 18, 20, 22 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values utilized by the PCU, its control module, and/or its associated modules during the period of time, and/or records physiological data collected during the period of time. During the infusion, physiological data associated with the patient is recorded within the session, operating parameter values, and modifications to the operating parameters of the PCU, its control module, and/or modules are also recorded in the session.

If not already logged into the PCU 12, the clinician may scan his or her badge proximate to a sensor (e.g., 54, 60) on the PCU 12, and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 14 (or its control module) to associate the patient with the PCU and/or module(s) (and a session).

The control unit 14 of PCU 12 is configured to generate a graphical representation of the infusion session, and display (e.g., in a display) the graphical representation, including a graphical visualization of all parameters of the infusion during the session and modifications to the parameters, together with physiological data obtained during the session. The graphical representation may include pseudo identifiers for unknown data until such data is substituted with known identifiers. At that time, the graphical representation is displayed with the known patient identifiers.

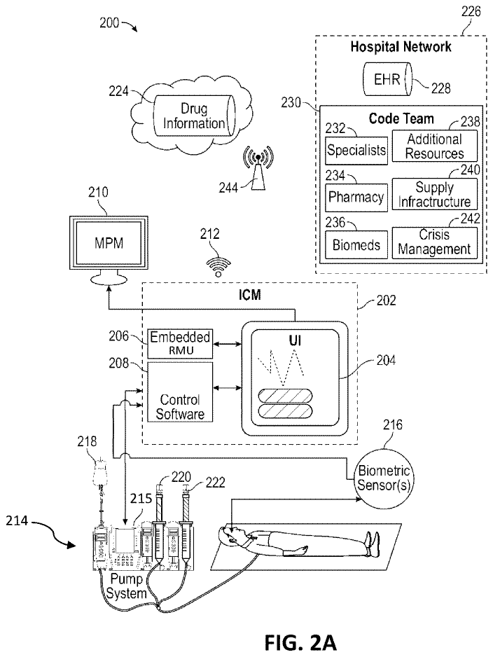

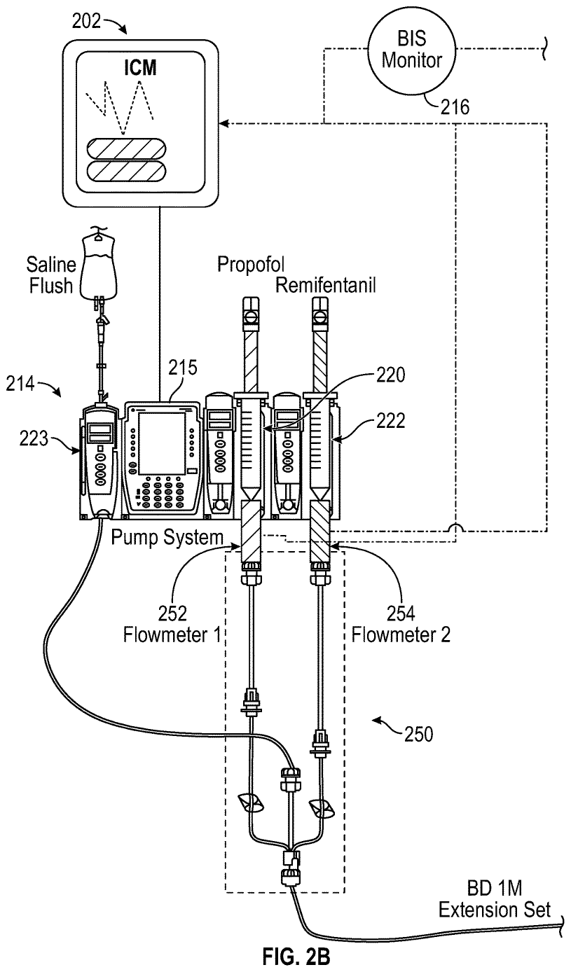

FIG. 2A is a conceptual diagram illustrating an example system architecture, including an example resource management unit, according to aspects of the subject technology. In the depicted example, a care provision system 200 includes an intelligent control module (ICM) 202. The ICM 202 (or infusion device control system) provides direct control over connected medical devices to assist the clinician in providing a more centralized control and management of the devices.

The ICM 202 provides a modular interface system by which various biometric sensors 216 used in a medical environment may be connected in a generic way to facilitate control over the connected medical devices. For example, the biometric sensors 216 may include one or more of a heart rate monitor, an oxygen sensor, and an intravenous (IV) flow rate monitor, all of which may be connected to the ICM 202 to facilitate, in addition to input by the clinician, centralized control of one or more infusion devices. As will be described further, the ICM 202 may be connected to a bi-spectral index (BIS) sensor 216 to assess the depth of anesthesia during an operation.

According to various implementations, the ICM 202 is a standalone device, separate from the infusion device(s). In some implementations, the ICM 202 is a mobile device with a form factor like a tablet computer. The ICM 202 may be portable with a rechargeable power source (e.g., a battery). In some implementations, the ICM 202 may be configured to integrate with (e.g., share power with) an infusion device 214. The ICM 202 may further connect to a server and/or cloud-based system for further data input, data coordination, and reporting. Examples of cloud-based systems include a cloud-based drug information database 224 (or formulary), and a hospital network 226 that includes electronic medical record (EMR) system or database 228.

The hospital network 226 may also include a code team 230 system and/or network that responds to code alarms. The code team 230 includes specialists 232 (human or AI), a pharmacy 234, biomedical technicians 236 (human or AI), crisis management resources 242, supply infrastructure 240, and additional resources 238 for responding to requests made to the code team 230.

In the depicted example, the ICM 202 includes a control unit 208, including one or more processors and/or control software and/or control algorithms. In some implementations, control unit 208 includes connectivity circuitry. In some implementations the control unit 208 includes software to enable closed and semi-closed-loop control capabilities over one or medical devices at a point of use. In some implementations, the ICM 202 provides an external interface, for example a user interface 204, for interaction between an IV infusion pump 214 and one or more different physiological or biometric sensors 216, and for providing input parameters that may be used to control the titration of IV infusions of medications to a patient.

In some implementations, the closed-loop system provides control for the IV infusion of the medication by an infusion device 214 according to the feedback provided by the biometric sensors 216 used to monitor the therapy being performed. In situations where the IV medication is not being delivered as a result of abnormal conditions such as the IV line being occluded, leaking, or not being connected to the patient, the sensors 216 will indicate that the patient is not responding to the therapy. The subject technology provides feedback (e.g., a notification or alarm) to the clinician to alert the clinician of a possible fault condition that should be addressed, in some instances before a critical situation occurs.

The ICM 202 can incorporate control software (including, e.g., one or more algorithms in the unit 208) that can be tailored to specific or general medical treatments. The ICM 202 may receive infusion status information from infusion device 214. The infusion status information may include, for example, an identification of the medication, a flow rate of an administration of medication to a patient by the infusion device, a VTBI (volume to be infused), delivery duration, upstream or downstream pressure, and the like. While the infusion device 214 may have its own safety system, the ICM 202 may be preprogrammed to determine safety events such as events specific to a particular procedure. The ICM 202 may determine, based on sensor data from sensor(s) 216 and infusion status information, that a safety event is likely to occur within a predetermined period of time (e.g., programmed into the ICM or determined by AI based on training models for the procedure being undertaken).

A closed-loop control system as described herein generally refers to a system that does not rely on external manual inputs to deliver a therapy. Once configured, the closed-loop system can autonomously provide a therapy, receive feedback from one or more sensors 216 and, based on the feedback, automatically adjust the therapy as needed. For example, the ICM 202 may determine, during an administration of a medication, an expected trend in a physiological property during a predetermined time period based on sensor data for a prior period of time, a dose of the medication provided to the patient, and the one or more physical parameters of the patient. The ICM 202 may then cause the infusion device to adjust the dose of the medication to cause the physiological property to follow an expected trend within a predetermined time period.

Having the ICM as a separate unit from the medical device provides several advantages. For example, the advancement of sensors (e.g., biometric sensor(s) 216) may be much more rapid than the development of infusion pump systems (e.g., pump device 214), and thus the control system may accommodate these changes more quickly. It may be desirable to update control algorithms to address changes in treatment methods, available medications, and patient physiology. For this reason, it may be desirable to have the algorithm reside in a component different than the pump system, in order to accommodate more frequent changes. Moreover, machine learning and artificial intelligence may account for patient variations related to physiological properties such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone, but may reside on a server 30 or other cloud-based system accessible from the ICM.

Additionally, the ICM 202 of the subject technology may be adaptable to a variety of pump systems and sensor inputs. The ICM 202 may further be configured to add wireless, Bluetooth and LAN connections to pump systems that do not currently have it available. For example, the FIG. 2 shows the ICM 202 having a wireless connection 212 for communication with a network system 244 at the hospital. Adding such communications to the pump system 214 may enable other capabilities such as remote monitoring and control of the infusion pumps, and facilitating access to EMR 228. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

According to various implementations, the ICM 202 facilitates the control software 208 to be separate from the embedded firmware 206 of the pumps and from the sensors, which can facilitate a scalable and rapidly configured system to provide closed-loop control of medical treatments. Moreover, the ICM 202 can be configured to operate with a multitude of sensors by way of electrical connectors and/or wireless communication. The ICM 202 may include one or more microprocessors and algorithms to provide signal conditioning and/or conversion of the sensor signals to the appropriate physiological properties for the connected medical device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

The user interface 204 of ICM 202 can include a display module or a touch-sensitive display module configured to provide a user interface for display of information pertaining to patient physiological status, as well as system control status. In some implementations, the user interface 204 includes circuitry within the ICM 202 housing that provides display information to an external display device. An example of such an external display device includes a multi-parameter monitor (MPM) 210. The MPM 210 may, for example, display various vital statistics (e.g., electrocardiogram saturation level (SpO2) of a patient in a surgery room such that the vital statistics are visible to the clinicians (e.g., all the clinicians) in the room. The display on the ICM 202 may be mirrored via an associated MPM 210 to provide information to devices connected to the MPM 210 and/or clinicians involved in the treatment.

The ability of the ICM 202 to connect to another display (e.g., the MPM 210) provides modular scalability. For example, a more extensive user interface may be beneficial in some use cases. The more extensive user interface may include displays of data and graphs, for which a larger high-resolution display may be used. Some use cases may benefit from a display that have minimal information and a corresponding user interface to support such a configuration. A smaller, space saving and/or lower cost locally connected display may then be used.

According to various implementations, the ICM 202 includes a resource management unit (RMU) 206. The RMU 206 stores protocol information (e.g., information about processes and steps associated with therapies and patients), making the information readily accessible in the ICM 202. In some implementations, the RMU 206 provides information to the user interface 204, allowing the system to more efficiently (e.g., using fewer device resources such as memory, power, processing cycles, user interface area, and the like) present and navigate through pertinent information (e.g., using touch inputs on a touch screen). According to variously implementations, the RMU 206 guides the user to the order of steps and information needed at the appropriate time thereby avoiding expenditures of resources on information that is untimely or irrelevant to the currently detected condition.

In some implementations, the ICM 202 may access additional resources through the communication capabilities of the RMU 206. For example, the RMU 206 may access additional resources relating to medication information and dosage calculations from an internal or remote storage (e.g., from the drug information database 224, from a centralized). In some implementations, the RMU 206 can cause the user interface 204, and/or other operably connected user devices to display a dosage calculator for a patient when a particular medication is to be administered. In some implementations, the dosage calculator is a weight-based calculator that accounts for a weight of the patient (e.g., the weight of the patient is provided to the ICM 202 by manual entry by a clinician, or based on information received from the EHR database 228). In some implementations, calculations for weight-based medications can be readily accessed through the user interface 204 or the one or more user devices communicatively connected to the ICM 202. A patient's electronic health record may also be readily accessed directly through the ICM 202 to provide critical health and allergy information to the clinicians. The additional resources relating to medication information accessed by the ICM 202 via the RMU 206 may include information about a compatibility of medications that a patient is prescribed to receive. In some implementations, the RMU 206 can cause the user interface 204, and/or other user devices to display mixing ratio instructions. For example, the RMU 206 may cause the user interface 204 to specify a current or programmed flow rate of the first fluid from the syringe pump 220, a current or programmed flow rate of the second fluid from the syringe pump 222, and a current or programmed flow rate of the infusion fluid from the infusion bag 218 to facilitate an appropriate mixture for administration to the patient during the infusion.

In one example, the RMU 206 may access additional resources by allowing a clinician to electronically deliver or make pharmacy requests via the ICM 202 to the pharmacy 234. The RMU 206 can cause the user interface 204 to display controls that allow a clinician to summon one or more specialists for consultation (e.g., virtually using cameras communicatively connected to the ICM 202 and/or data from the biometric sensors 216; or request for an in-person consultation at the patient's location). In some implementations, the RMU 206 may connect the clinician to an AI. The RMU 206 can cause the user interface 204 to display controls that allow a clinician to send code alarms so that additional resources and personnel (e.g., human or AI) can respond to a medical issue. The RMU 206 can cause the user interface 204 to display controls for equipment requests or for other resources needed by the patient. Thus, in the cases where a hospital code would need to be issued, a clinician can simply use the RMU 206 to bring up checklists that would then present appropriate codes available for specific requests to be sent via the hospital network 244. In some implementations, the user interface may include a control element that, when activated, transmits a control message to a device in communication with the RMU 206 to request a resource.

FIG. 2B illustrates the example system architecture of FIG. 2A with multiple infusion sets providing medications to a single extension set, according to aspects of the subject technology. In the depicted example, two infusion pumps 220 and 222 are connected to a patient control unit 214 of an infusion device 214. Pumps 220 and 222 are connected to one or more infusion sets, such as a multi-connector set 250 (e.g., a Y connector), which then provides a combination of medications to the patient via an IV administration line connected to a single intravenous cannula.

The depicted multi-connector set 250 is connected to a first syringe containing propofol and a second syringe 222 administering remifentanil. A first flow meter 252 (e.g., inserted into the line) monitors a flow of the medication from the first syringe 220 and a second flow meter 254 monitors a flow of the medication from the second syringe 222. A third connector of the multi-connector set 250 is shown connected to a third pump 223 which administers a diluent such as saline. In some implementations, the multiple medications may flow into a single extension set, and each pump 220, 222, 223 may be controlled electronically by the patient control unit 215 to provide a precise admixture for even administration to the patient.

The flow sensors 252, 254 can be used to provide verification of the delivery of the medication. This provides the benefit of additional feedback to the control software 208 which can be used to better control the administration of the medication and help to reduce the usage. It would also provide record keeping verification to prevent diversion of the medications. The flow sensors can be of the thermal or other electromechanical types that would connect directly to the ICM 202.

The system administers the medication using two syringe pumps each controlling the specific medication. Anesthetists often use the multi-lumen or Y connectors to allow infusion of the different anesthetic agents with or without other intravenous fluids to be given through a single intravenous cannula. According to various implementations, the RMU 206 stores operating procedures and procedure workflows for the operating procedures, including sequencing of medication use. The ICM 202 is connected to the infusion device 214 via the patient control unit 215 and electronically communicates with the patient control unit to monitor the sequencing of the operations programmed into multiple pumps during the procedure, including parameter changes (e.g., to flow rate, VTBI, etc.). Flow rate may vary independent of clinician actions.

In some implementations, the ICM 202 may monitor for occlusions across multiple pump channels and adjust pump operations of occluded channels and/or non-occluded channels to ensure patient and pump safety. In the depicted example, flow rates from each pump 220, 222 are provided by flow sensors 252, 254 to the ICM 202, which then acts as a buffer between the flow sensor(s) and other sensors 216 and the patient control unit 215. In this manner, the ICM 202 intercepts signals that would normally be provided directly to the patient control unit 215 so that the ICM 202 may act as a watch-dog to detect flow events and may, depending on stored operating procedures and data received from connected sensor(s) 216, adjust the flow rate and/or sensor data before it is provided to the patient control unit 215. In this regard, the patient control unit 215 requires no modification and continues to operate according to its current programming, while the ICM 202 may be used by the clinician to make adjustments and/or to expand functionality of the infusion device 214.

When a first pump is stopped to change a medication container, the ICM 202 may cause a second pump to alter operations, or sequencing of pump operations between multiple pumps may be re-sequenced, so that the patient is not adversely impacted. For example, if one pump is nearing empty and the second pump is operating at high flow rate, the system may slow the high flow rate pump to avoid a bolus delivery by the high flow rate pump. In some implementations, the ICM 202 may make adjustments based on received parameters such as the type or size of a connected infusion set, the length of the infusion line, the type of therapy being implemented, type of drug, head height, flow rate(s) (e.g., from sensors 252, 254), and/or the like. In some implementations, a sensor may be included on the line to detect type of substance flowing through the set (e.g., similar set up to the flow meters shown in figure above). In this manner the ICM 202 may perform a quick result to confirm the line is connected to the correct medication, for example, by performing a pH test, conductivity, spectral, optical, acoustic, or other measured property on the fluid. The ICM 202 may prevent the infusion device or other system component from running without detecting the approved medication or infusion set, or until an override by the clinician is received.

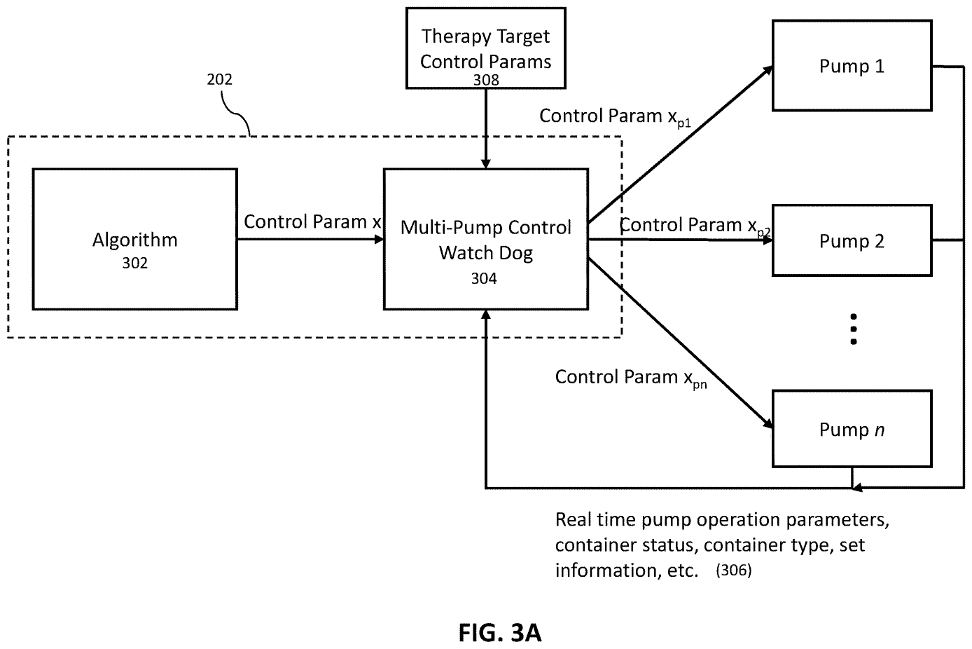

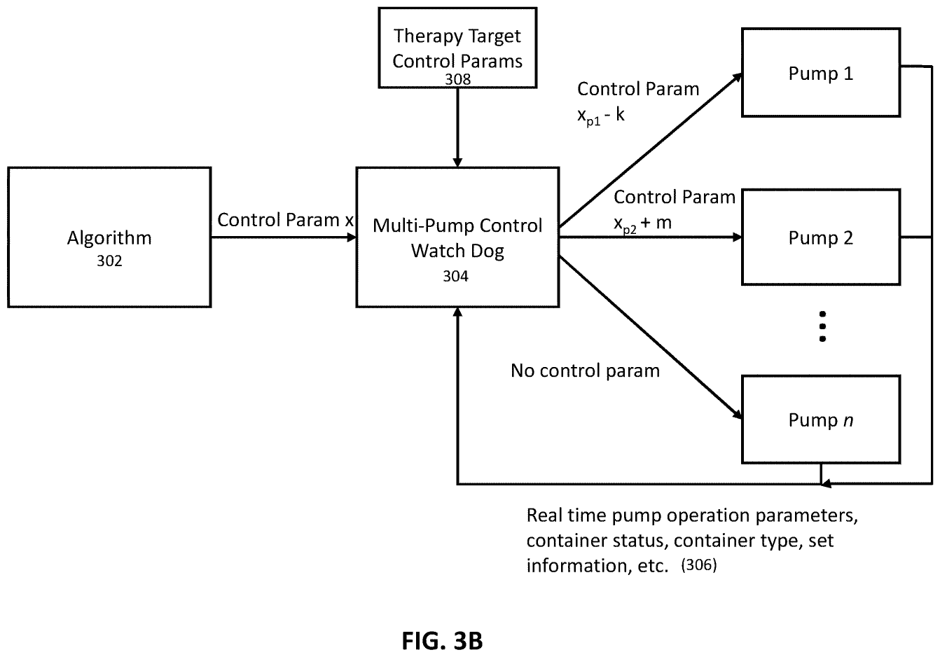

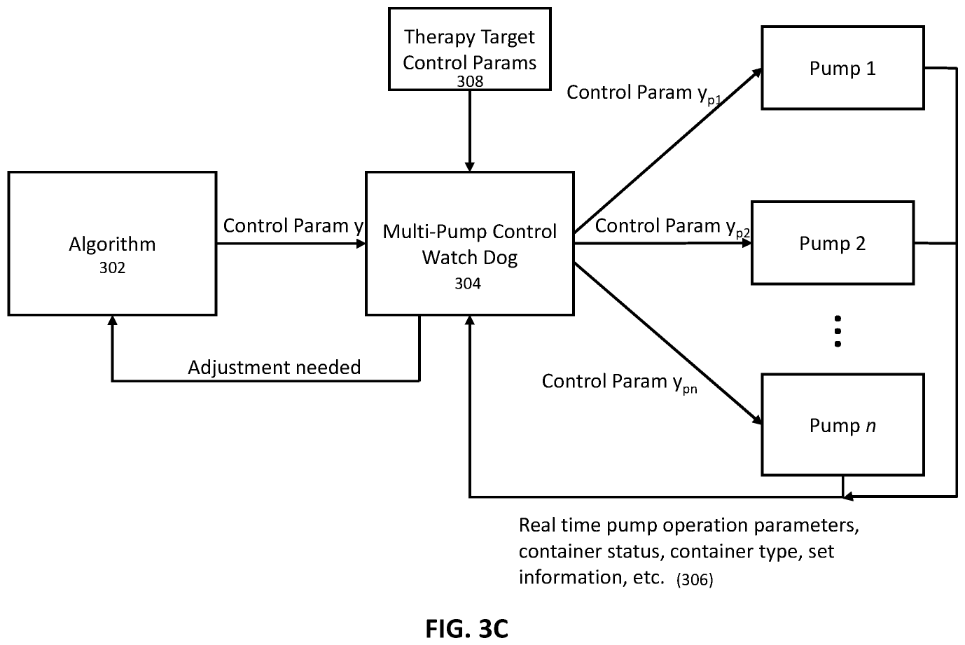

FIG. 3A is a first example conceptual diagram for managing multiple infusion pumps using the intelligent control module in a closed-loop or semi-closed-loop environment, according to various aspects of the subject technology. In the depicted example, the ICM 202 includes a control algorithm 302 and a multi-pump control watch-dog algorithm 304. In this regard, the ICM 202 is operably connected to each pump p1-pn (e.g., by one or more respective patient control units 215) and is configured to control each pump by adjusting the pumps' internal operational parameters. As depicted, the ICM 202 (and/or algorithms) monitors real-time infusion information 306 such as operational parameters, container type, set information, infusion status (e.g., container status), and the like and, based on this information readjust the control parameters as needed to provide a therapy to the patient. Though not shown in FIG. 3A, the ICM 202 may additionally or alternatively receive real-time sensor information associated with the patient to readjust the control parameters needed to provide the therapy. According to various implementations, a clinician or other system (e.g., EMR system) may provide further parameters 308 to the ICM 202 to facilitate the monitoring and/or control of the connected pumps.

As used herein, “real-time” may refer to availability for processing at or near in time to the time the associated item is generated or detected. The algorithm(s) 302 may be stored locally in a memory of ICM 202 or, in some implementations, downloaded dynamically from a server 30 and/or database 37. In some implementations, the algorithm(s) 302 may include derived input values from monitoring or lab results and the like. The algorithm(s) 302 may use advanced processing capabilities such as artificial intelligence, machine learning, and neural net processing.

As described with regard to FIG. 1, infusion devices 214 may include modular infusion platforms that are expandable with multiple medication delivery modules (e.g., modules 16, 18, 20, 22, 220, 222) to handle more than one type of medication delivery to a patient. Different patients require different levels of treatment, and different parameters for being treated by different devices. Each individual infusion module, and different types of infusion devices generally, may operate differently, resulting in a wide variety of parameter variations (and interface possibilities) that may confuse or distract clinicians during infusion and other medication delivery procedures. During surgery and under conditions where direct access to the infusion device or devices may be difficult, precise control and coordination of the devices may also be difficult. Failure to maintain control over the devices increases the health risk to the patients. For example, under manual control, the amount of time a patient is provided medication at a level that is within a desirable range can fluctuate based on, for example, the attentiveness of the clinician. The amount of time a patient spends outside the desired range can have an impact on the procedure, patient, recovery, and resources used for administering the therapy. Furthermore, some therapies require repetitive work steps that can be prone to error or complacency, which may lead to an adverse event. With reference to both FIG. 2A and FIG. 3A, in operation, the ICM 202 operably connects to multiple medical devices and, upon recognizing the medical device, the user interface software determines one or more predetermined therapies associated with the medical device. For example, the software may receive an identifier of the medical device upon connection to an infusion device 214 and then query (e.g., over network 10) database 37 for the available therapies associated with the infusion device. A selection control may then provide the therapies to the clinician for selection. When a selection of a therapy is made, the system determines which sensor device(s) 204 associated with the therapy are operably connected to the ICM 202 and identifies and configures the corresponding algorithm(s) 302, 304 for operation of the connected medical device(s) based on real-time data received from the connected sensor(s) 216.

Each pump p1-pn is configured to accept programming from the ICM 202. In this manner, a user of the ICM 202 may directly program a respective pump by selecting the pump via the user interface 204 and select one or more parameters to set or adjust. The ICM 202 may then provide the parameter(s) to the pump and the pump modifies its operation accordingly. According to various implementations, responsive to the receiving infusion related information 306, the ICM 202, operating with watch-dog algorithm 304, may automatically adjust pump parameters Xₚ₁-Xₚₙ for the respective pumps p1-pn.

In some implementations, the control parameters may be set by a clinician and so long as the therapy is proceeding as planned and there are no unexpected results (e.g., out of bounds readings) the control parameters may remain stable. A control parameter x is received from the closed-loop algorithm and passed to each respective pump unchanged, or adjusted and the same adjusted value is passed to each pump.

In some implementations, the watch-dog algorithm 304 may be configured to convert control parameters for a single therapy into control parameters for multiple therapies. For example, the control algorithm 302 may be configured to control administration of a single medication from a single pump, and may provide a control parameter x for administration of a single medication X to the patient. The watch-dog algorithm 304 may be configured to control administration of multiple medications based on the parameters for medication X. In this regard, the watch-dog algorithm 304 may convert control parameter x to control parameters xₚ₁, xₚ₂, . . . xₚₙ and pass the control parameters to respective pumps pl-pn. Based on the therapy configuration selected by the clinician via the user interface 204, Algorithm 304 may be configured to change a given parameter by a given amount for each pump. For example, a weight may be given to each medication of each pump based on pharmacokinetic profiles of the medications provided by the pumps, and algorithm 304 may perform, for each pump, a predetermined weighted adjustment to the parameter x before providing a parameters xₚ₁, xₚ₂, . . . xₚₙ to each pump.

As an example, the control-loop algorithm 302 may be configured to maintain a patient in anesthesia using propofol. However, the clinician may select to administer both propofol and remifentanil to the patient. The watch-dog algorithm 304 implemented by the ICM 202 may be configured to control two pumps to administer propofol and remifentanil based on the single control parameter x provided by the control-loop algorithm 302 (e.g., for propofol). In this regard, the ICM 202 (and watch-dog algorithm 304) may be used to repurpose control-loop algorithm 302 without change to the original algorithm.

According to various implementations, the ICM 202 includes alarm software. In some situations, a fault, failure, or attention needed at a medical device or sensor connected to the ICM 202 may generate one or more alarms. In cases where multiple devices are connected to the ICM, alarms that may not need immediate attention (e.g., a warning that the syringe is nearing empty), a hierarchy can be employed providing information on what needs to be addressed. The notification can be provided by means other than an audio alarm which would distract all clinicians involved in the surgery.

The algorithm 302 may also monitor all connected devices to predict potential faults prior to happening so that a clinician may act on them in a timely manner before a situation occurs requiring immediate action. In some implementations, the algorithm 302 may combine information from the various sensors to identify a problem or a potential for a problem. For example, in the case of anesthesia, if a patient's bi-spectral index sensor 216 (BIS) is showing a rise in the BIS value together with changes in hemodynamic parameters, and a flow sensor 250 and/or sensor(s) within the infusion device 214 indicate that the rise in the BIS value is inversely proportional to the administration of drugs being infused, the ICM 202 may indicate that the patient is not responding as expected from receiving the medication. In such implementations, a hierarchy of potential faults can be displayed on the ICM 202 providing guidance to the clinician of what steps that need to be taken to bring the therapy safely into compliance (e.g., correspond to target therapy parameters). For example, the fault code on the user interface 204 may appear with an indication of one or more potential causes. The ICM 202 may also display a fault checklist showing potential checks to perform such as checking that the IV line is connected, not obstructed or the sensors are connected.

As another example, anesthesia may require a predetermined amount of an administration of drugs to achieve the required end points of hypnosis, immobility, and suppression of reflexes during surgery. It may be given as the combination of a hypnotic and an opioid, with the anesthesiologist manually titrating doses or infusion rates of the 2 drugs to provide the best balance. Using a bi-spectral index sensor 216 (BIS), the ICM 202 may monitor the depth of anesthesia and remotely control the infusion device to administer a proper amount of an IV drug(s), while preventing awareness or excessive anesthetic depth during medical treatment, thereby improving patients' outcomes. As described previously, the ICM 202, when configured, may adapt a single pump solution into a multi-pump solution to deliver the hypnotic and the opioid based on a predetermined algorithm for delivery of one of the drugs.

As another example, the ICM 202 may be connected to a sensor configured as a blood glucose monitor and, based on intelligent modeling and continuous blood glucose measurements, maintain a patient's blood glucose by way of controlling an infusion device's administration of insulin and/or dextrose solution(s). Blood glucose (BG) disorders, such as stress-induced hypoglycemia and hyperglycemia, can be common complications in patients in the ICU. In addition, patients with type 1 and 2 diabetes may be susceptible to hyperglycemia, as well as severe hypoglycemia as a result of overcorrection with insulin. In one example, IV infusions of insulin and/or dextrose may be controlled by the ICM 202 using a closed-loop configuration with inputs from continuous BG measurements to maintain BG levels within the desired range.

FIG. 3B is a second example conceptual diagram for managing infusion pumps using the intelligent control module in a closed-loop or semi-closed-loop environment, according to various aspects of the subject technology. In the depicted example, control parameters provided to the connected pumps are dynamically adjusted based on real time therapy information provided by the pumps.

As described previously, the ICM 202 may configured in a feedback loop with multiple pumps. In this regard, the pumps p1-pn are configured to accept programming from the ICM 202 while providing infusion information to the ICM 202 as a form of feedback. According to various implementations, the algorithm 302 may operate the pumps in a closed-loop based on infusion information 306 received from the pumps as well as sensor information provided by connected sensors 216, therapy target control parameters 308 and/or patient or medication information provided by a clinician via the user interface 204 or from external systems such as a hospital information system 30 (e.g., an EMR server).

In the depicted implementation, the algorithm 302 is a closed-loop algorithm. The closed-loop algorithm 302 may be loaded on ICM 202 or may provide control parameters from an external system such as the hospital information system 30. In this regard, the closed-loop algorithm 302 may be a static algorithm, and ICM 202 (including, e.g., a watch-dog algorithm) may be configured between the closed-loop algorithm 302 and the pump(s) it controls. In operation, the ICM 202 intercepts commands provided to one or more pumps pl-pn by the closed-loop algorithm 302 and controls the pumps based on the commands as well as real-time operating parameters and infusion status information 306 from the pumps, and the therapy target control parameter 308. The ICM 202 modifies the intercepted commands (e.g., based on the real-time operating parameters and infusion status information 306 and the therapy target control parameter 308) to control a therapeutic effect of the therapies provided by the pumps, without input to the closed-loop algorithm.

In the depicted example, the ICM 202 receives a control parameter x from the closed-loop algorithm 302. Based received real-time operating parameters and infusion status information 306 and the therapy target control parameter 308, the ICM 202 determines that, instead of passing the control parameter to the pump(s), the control parameter should be adjusted differently for each pump. For example, the ICM 202 may determine that pump n is occluded, or the medication container is empty. Accordingly, the ICM 202 may determine that the control parameter value should be reduced before being sent to a first pump pl and increased before being sent to a second pump p2. No further parameters may be sent to pump n, or pump n may be instructed to terminate the infusion, enter a standby mode, power down, and/or provide an alarm.

In another example, the therapy provided by the pumps may be an induced state of anesthesia, and sensor information received by the ICM 202 may include electroencephalographic activity of a patient to which the first and second therapies are provided (e.g., from sensor 216). The first pump p1 may administer a first anesthesia drug and the second pump p2 may administer a second anesthesia drug. Responsive to the electroencephalographic activity (and other infusion information 306 or target parameters 308), the ICM 202 (by programming the pumps) may cause a decrease in a flow of the first anesthesia drug while causing an increase in the flow of the second anesthesia drug. A third drug or fluid provided by a third pump may be terminated, not adjusted, or the control parameter may not be sent to the third pump.

FIG. 3C is a third example conceptual diagram for managing infusion pumps using the intelligent control module in a closed-loop or semi-closed-loop environment, according to various aspects of the subject technology. In some implementations, the watch-dog algorithm 304 may not modify the control parameters received from the closed-loop algorithm but, rather, may instruct the closed-loop algorithm that an adjustment is required. The instruction may include a code or other information indicating an event causing the need for an adjustment (e.g., occlusion, alarm, patient harm, etc.). The instruction may include information identifying a source of the event (e.g., which pump, which medication, etc.). In some implementations, the instruction may include the therapy target violated. In some implementations, the instruction may include a suggested adjustment specific to a device (e.g., rate for specific pump or drug) or generally applicable to the therapy (e.g., total infusion volume). The algorithm 302 may be configured to receive adjustments, for example, in the form of parameter value changes. If the watch-dog algorithm 304 determines that pump n is occluded or that the corresponding medication container is empty the watch-dog algorithm may not make changes but, rather, alerts the algorithm to the error or deviation condition. The control algorithm 302 then makes the adjustment, and the watch-dog passes the adjusted parameter to each of the pumps. As described previously, the watch-dog algorithm may make static or weighted adjustments prior to forwarding the parameter(s) to the pumps.

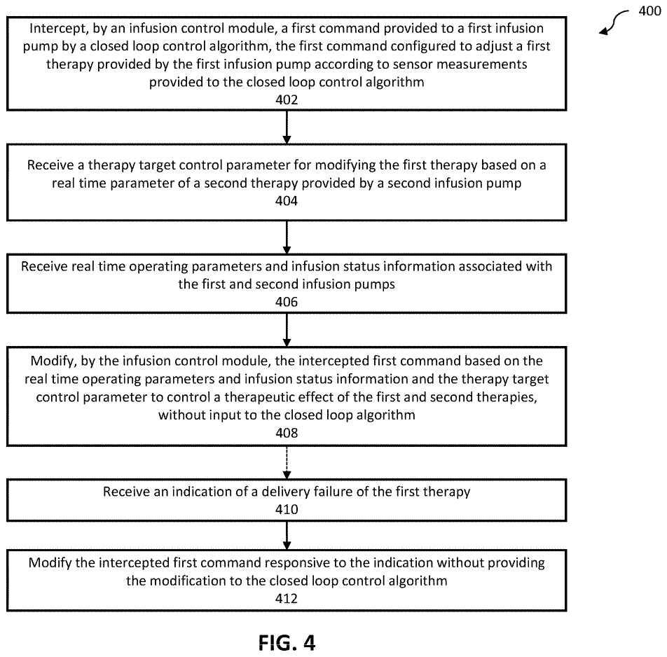

FIG. 4 depicts an example process 440 for managing multiple infusion pumps using an intelligent control system in a closed-loop or semi-closed-loop environment, according to various aspects of the subject technology. For explanatory purposes, the various blocks of example process 440 are described herein with reference to FIGS. 1 through 3C, and the components and/or processes described herein. The one or more of the blocks of process 400 may be implemented by a control device such as, for example, ICM 202 or a component thereof, interface 14, or one or more associated computing devices. As previously described, the example process 400 may operate under control of AI, which may analyze sensor data and make any of decisions within example process 400 on behalf of the clinician. If a hard decision cannot be made (e.g., certain thresholds not being met) then the ICM 202 (including, e.g., a processor such as the RMU 206) may suggest a soft decision to the clinician who may then confirm, reject, or change the selection to treat the patient.

According to the depicted example process 400, the ICM 202 provides an infusion communication system for managing multiple infusion pumps in a closed-loop or semi-closed-loop environment. A watch-dog control algorithm 304 of the ICM 202 intercepts a first command provided to a first infusion pump by a closed-loop control algorithm 302 (402). As described previously, the first command may be configured to adjust a first therapy provided by the first infusion pump according to sensor measurements provided to a closed-loop control algorithm 302.

For example, the closed-loop control algorithm 302 may be configured to automatically adjust one or more infusion devices and/or pumps based on information such as the sensor measurements provided by one or more sensors 216 associated with a patient, sensors (e.g., flow sensor 250, 252) associated with the pump(s), and infusion information provided by the infusion pump(s). For example, the algorithm 302 may be configured to maintain the patient at a particular therapeutic state (e.g., a hypnotic or sleep state) by controlling an amount of medication delivered by the pump(s) and to dynamically adjust the amount based on monitoring the patient with the sensors 216 for indications of how the patient is responding to the medication and/or how well the patient is stable within the therapeutic state.

In the depicted process 400, the ICM 202 receives a therapy target control parameter 308 for assessing and modifying the first therapy based on a real time parameter of a second therapy provided by a second infusion pump (404). As described previously, the first therapy may include a first administration of a first medication to the patient, and the second therapy may include a second administration of a second medication to the patient. The therapy target control parameter 308 may be an operating range for a sensor measurement (e.g., a bi-spectral index range) or a target for the sensor measurement. For example, the ICM 202 may control the pumps, including modifying control parameters (x1-xn) received from the closed-loop algorithm 302 to maintain the patient is a state consistent with the target. In this regard, a target control parameter may be a threshold such as a flow rate or pressure threshold associated with one or more of the connected pumps, or may be an upper or lower limit for a parameter.

The ICM 202 receives infusion information 306, including real time operating parameters and infusion status information associated with the first and second infusion pumps (406). According to various implementations, the infusion information may be received by the control-loop algorithm 302 or the watch-dog algorithm 304, or both.

The ICM 202 modifies the intercepted first command based on the infusion information 306 (e.g., the real time operating parameters and infusion status information) and the therapy target control parameter 308 to control a therapeutic effect of the first and second therapies, without input to the closed-loop algorithm (408). In this example, while the closed-loop algorithm 302 may receive feedback from the various pumps and sensors, the algorithm does not receive any other extraneous input from the ICM 202 that pertains to or is associated with the modification. In other words, in some implementations, the modification is performed without the knowledge of, and independent of, the closed-loop algorithm 302.

In some implementations, the therapeutic effect is a predetermined hypnotic state of anesthesia, and the sensor measurements include electroencephalographic activity of a patient to which the first and second therapies are provided. In some implementations, the therapeutic effect includes a stable cardiac output, and the sensor measurements comprise a measure of the patient's cardiac output.

In one example, the ICM 202 receives an indication that a container for the second medication is nearing empty and that the second infusion pump is operating at a high flow rate. In this regard, the ICM 202 may cause, responsive to the indication, the second infusion pump to slow a flow rate of the second administration. In this regard, the patient may be prevented from receiving a bolus of the second medication as the first medication empties.

In another example, the ICM 202 monitors commands sent by the closed-loop control algorithm to the first and second infusion pumps. The ICM 202, based on the monitoring, intercepts a second command prior to or contemporaneous with intercepting the first command, and determines that the first command should be processed prior to the second command to control the therapeutic effect. In this example, the ICM 202 delays the second command from being provided to the second infusion pump until the first command is adjusted and provided to the first infusion pump.

In another example, the first therapy includes a first administration of a first anesthesia drug, and the second therapy comprises a second administration of a second anesthesia drug. The ICM 202 determines a decrease in a flow of the second administration and, in response, increases the flow of the first administration to maintain the patient in the predetermined hypnotic state of anesthesia based on the decrease in the flow of the second administration. As described with other implementations, the increase is not provided to the closed-loop control algorithm.

In some implementations, the ICM 202 (optionally) receives an indication of a delivery failure of the first therapy (410). The indication of the delivery failure may, for example, be received based on the infusion status information. For example, a pump may provide failure status code as part of the status information it reports to the ICM 202 (and, e.g., other hospital systems).

The ICM 202 (optionally) modifies the intercepted first command responsive to the indication of delivery failure without providing the modification to the closed-loop control algorithm (412).

In some implementations, the ICM 202 detects, via a sensor 250, 252 operably connected to an infusion line of the first therapy, a type of fluid flowing through the infusion line, and the ICM 202 prevents, based on intercepting commands sent to the first and second infusion pumps 220, 222, the first infusion pump from providing the first therapy or the second infusion pump from providing the second therapy until the detected type of fluid is confirmed for the first and second therapy.

In some implementations, the ICM 202 identifies an infusion set used for the first therapy (e.g., by the set being scanned by a scanner or by RFID detection), and prevents, based on intercepting commands sent to the first and second infusion pumps, the first infusion pump from providing the first therapy or the second infusion pump from providing the second therapy until the infusion set is confirmed for the first and second therapy.

In some implementations, based on infusion information associated with the second infusion pump, the ICM 202 determines that an infusion line of the second infusion pump is occluded, and modifies the intercepted first command to cause a flow rate of the first infusion pump to be adjusted without providing the adjusted flow rate to the closed-loop algorithm.

Many of the above-described example steps of processes 400 and 440, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

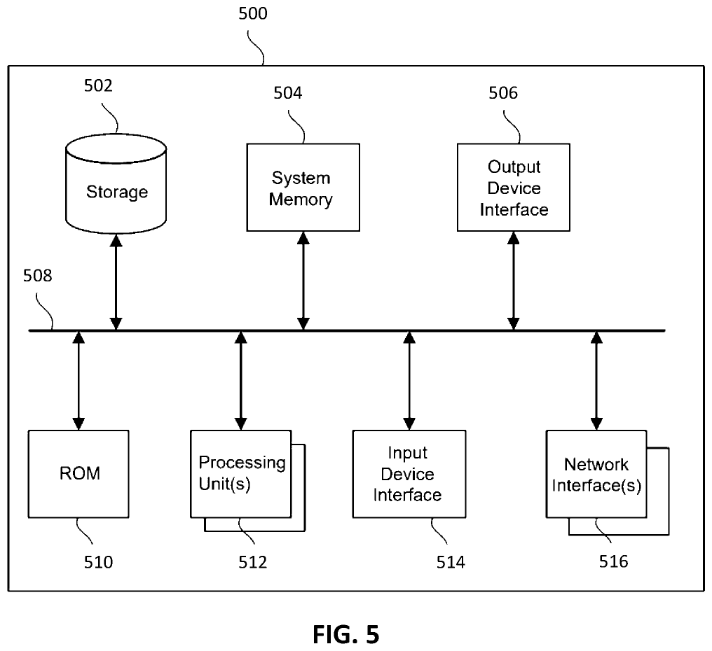

FIG. 5 is a conceptual diagram illustrating an example electronic system 500 for managing multiple infusion pumps using an intelligent control system in a closed-loop or semi-closed-loop environment, according to aspects of the subject technology. Electronic system 500 may be a specifically configured computing device for execution of software associated with one or more portions or steps of the components and processes provided by FIGS. 1 through 4, including but not limited to the ICM 202, information system server 30, database 37, computing hardware within patient care device 12, or a remote device 32 (e.g., a mobile device). Electronic system 500 may be representative, in combination with the disclosure regarding FIGS. 1-4. In this regard, electronic system 500 may be a specifically configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506 specifically configured to perform one or more of the infusion features described. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure. These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include specifically configured electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to specifically configured microprocessor or multi-core processors that execute software, some implementations are performed by one or more similarly configured integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices configured to implement one or more of the infusion features described. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A method comprising, by an infusion device control system: intercepting a first command provided to a first infusion pump by a closed-loop control algorithm, the first command configured to adjust delivery of a first fluid by the first infusion pump according to sensor measurements provided to the closed-loop control algorithm; receiving a therapy target control parameter for modifying the delivery of the first fluid based on a real time parameter for delivering a second fluid by a second infusion pump; receiving, for the first and second infusion pumps, real time operating parameters and infusion status information; and modifying the intercepted first command based on the real time operating parameters and infusion status information and the therapy target control parameter to control the deliveries of the first and second fluids.

Clause 2. The method of Clause 1, further comprising: receiving, based on the infusion status information, an indication of a delivery failure of the first fluid; and modifying the intercepted first command responsive to the indication of the delivery failure.

Clause 3. The method of Clause 1 or Clause 2, further comprising: receiving an indication that a container for the second fluid is nearing empty and that the second infusion pump is operating at a high flow rate; and causing, responsive to the indication, the second infusion pump to slow a flow rate of the delivery of the second fluid.

Clause 4. The method of any one of Clauses 1-3, further comprising: determining, based on the infusion status information of the second infusion pump, that an infusion line of the second infusion pump is occluded, modifying the intercepted first command to cause a flow rate of the first infusion pump to be adjusted.

Clause 5. The method of any one of Clauses 1-4, further comprising: monitoring, by an infusion communication system, commands sent by the closed-loop control algorithm to the first and second infusion pumps; intercepting, by the infusion communication system based on the monitoring, a second command prior to or contemporaneous with intercepting the first command; determining that the first command should be processed prior to the second command to control the delivery of at least one of the first and second fluids; and delaying the second command from being provided to the second infusion pump until the first command is adjusted and provided to the first infusion pump.

Clause 6. The method of any one of Clauses 1-5, wherein delivery of the first and second fluids cause an effect, wherein the effect is a predetermined hypnotic state of anesthesia, and the sensor measurements comprise electroencephalographic activity of a patient to which the first and second fluids are delivered.

Clause 7. The method of Clause 6, wherein the first infusion pump is configured to delivery a first anesthesia drug, and the second infusion pump is configured to deliver a second anesthesia drug, wherein the method further comprises: determining a decrease in a flow of the delivery of the second anesthesia drug, and increasing the flow of the delivery of the first anesthesia drug to maintain the patient in the predetermined hypnotic state of anesthesia based on the decrease in the flow of the delivery of the second anesthesia drug, wherein the increase in the flow is not provided to the closed-loop control algorithm.

Clause 8. The method of any one of Clauses 1-7, wherein delivery of the first and second fluids cause an effect, wherein the effect comprises a stable cardiac output, and the sensor measurements comprise a measure of the patient's cardiac output.

Clause 9. The method of any one of Clauses 1-8, further comprising: detecting, via a sensor operably connected to an infusion line of the first fluid, a type of the first fluid flowing through the infusion line; preventing, by an infusion communication system based on intercepting commands sent to the first and second infusion pumps, the first infusion pump from delivering the first fluid or the second infusion pump from delivering the second fluid until the detected type of the first fluid is confirmed as being scheduled for delivery by the first and second infusion pumps.

Clause 10. The method of any one of Clauses 1-8, further comprising: identifying an infusion set used for the first fluid; and preventing, by an infusion communication system based on intercepting commands sent to the first and second infusion pumps, the first infusion pump from delivering the first fluid or the second infusion pump from delivering the second fluid until the infusion set is confirmed as being scheduled for delivery by the first and second infusion pumps.

Clause 11. An infusion device control system, comprising: an infusion device comprising at least one of the first and second infusion pumps; at least two connection ports configured to communicatively connect to the infusion device and the sensor; and wherein the infusion device control system is configured to perform a method according to any one of Clauses 1 through 10.

Clause 12. A system, comprising: one or more processors; a memory device comprising non-transitory computer-readable instructions that, when executed by the one or more processors, cause the one or more processors to perform a method according to any one of Clauses 1 through 10.

Clause 13. A memory device comprising non-transitory computer-readable instructions that, when executed by one or more processors, cause the one or more processors to perform a method according to any one of Clauses 1 through 10.

Clause 14. A method comprising, by an infusion device control system: intercepting a first command provided to a first infusion pump by a closed-loop control algorithm, the first command configured to adjust a first therapy provided by the first infusion pump according to sensor measurements provided to the closed-loop control algorithm; receiving a therapy target control parameter for modifying the first therapy based on a real time parameter of a second therapy provided by a second infusion pump; receiving, for the first and second infusion pumps, real time operating parameters and infusion status information; and modifying the intercepted first command based on the real time operating parameters and infusion status information and the therapy target control parameter to control a therapeutic effect of the first and second therapies.

Clause 15. The method of Clause 14, further comprising: receiving, based on the infusion status information, an indication of a delivery failure of the first therapy; and modifying the intercepted first command responsive to the indication of the delivery failure.

Clause 16. The method of Clause 14, wherein the first therapy comprises a first administration of a first medication to a patient, and the second therapy comprises a second administration of a second medication to the patient, wherein the method further comprises: receiving an indication that a container for the second medication is nearing empty and that the second infusion pump is operating at a high flow rate; and causing, responsive to the indication, the second infusion pump to slow a flow rate of the second administration.

Clause 17. The method of Clause 14, further comprising: determining, based on the infusion status information of the second infusion pump, that an infusion line of the second infusion pump is occluded, modifying the intercepted first command to cause a flow rate of the first infusion pump to be adjusted.

Clause 18. The method of Clause 14, further comprising: monitoring, by an infusion communication system, commands sent by the closed-loop control algorithm to the first and second infusion pumps; intercepting, by the infusion communication system based on the monitoring, a second command prior to or contemporaneous with intercepting the first command; determining that the first command should be processed prior to the second command to control the therapeutic effect; and delaying the second command from being provided to the second infusion pump until the first command is adjusted and provided to the first infusion pump.

Clause 19. The method of Clause 14, wherein the therapeutic effect is a predetermined hypnotic state of anesthesia, and the sensor measurements comprise electroencephalographic activity of a patient to which the first and second therapies are provided.

Clause 20. The method of Clause 19, wherein the first therapy comprises a first administration of a first anesthesia drug, and the second therapy comprises a second administration of a second anesthesia drug, wherein the method further comprises: determining a decrease in a flow of the second administration, and increasing the flow of the first administration to maintain the patient in the predetermined hypnotic state of anesthesia based on the decrease in the flow of the second administration, wherein the increase in the flow is not provided to the closed-loop control algorithm.

Clause 21. The method of any one of Clauses 14-20, wherein the therapeutic effect comprises a stable cardiac output, and the sensor measurements comprise a measure of the patient's cardiac output.

Clause 22. The method of any one of Clauses 14-21, further comprising: detecting, via a sensor operably connected to an infusion line of the first therapy, a type of fluid flowing through the infusion line; preventing, by an infusion communication system based on intercepting commands sent to the first and second infusion pumps, the first infusion pump from providing the first therapy or the second infusion pump from providing the second therapy until the detected type of fluid is confirmed for the first and second therapy.

Clause 23. The method of any one of Clauses 14-21, further comprising: identifying an infusion set used for the first therapy; and preventing, by an infusion communication system based on intercepting commands sent to the first and second infusion pumps, the first infusion pump from providing the first therapy or the second infusion pump from providing the second therapy until the infusion set is confirmed for the first and second therapy.

### Further Considerations:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. A method comprising, by an infusion device control system:
intercepting a first command provided to a first infusion pump by a closed-loop control algorithm, the first command configured to adjust delivery of a first fluid by the first infusion pump according to sensor measurements provided to the closed-loop control algorithm;
receiving a therapy target control parameter for modifying the delivery of the first fluid based on a real time parameter for delivering a second fluid by a second infusion pump;
receiving, for the first and second infusion pumps, real time operating parameters and infusion status information; and
modifying the intercepted first command based on the real time operating parameters and infusion status information and the therapy target control parameter to control the deliveries of the first and second fluids.

2. The method of claim 1, further comprising:
receiving, based on the infusion status information, an indication of a delivery failure of the first fluid; and
modifying the intercepted first command responsive to the indication of the delivery failure.

3. The method of claim 1, further comprising:
receiving an indication that a container for the second fluid is nearing empty and that the second infusion pump is operating at a high flow rate; and
causing, responsive to the indication, the second infusion pump to slow a flow rate of the delivery of the second fluid.

4. The method of claim 1, further comprising:
determining, based on the infusion status information of the second infusion pump, that an infusion line of the second infusion pump is occluded,
modifying the intercepted first command to cause a flow rate of the first infusion pump to be adjusted.

5. The method of claim 1, further comprising:
monitoring, by an infusion communication system, commands sent by the closed-loop control algorithm to the first and second infusion pumps;
intercepting, by the infusion communication system based on the monitoring, a second command prior to or contemporaneous with intercepting the first command;
determining that the first command should be processed prior to the second command to control the delivery of at least one of the first and second fluids; and
delaying the second command from being provided to the second infusion pump until the first command is adjusted and provided to the first infusion pump.

6. The method of claim 1, wherein delivery of the first and second fluids cause an effect, wherein the effect is a predetermined hypnotic state of anesthesia, and the sensor measurements comprise electroencephalographic activity of a patient to which the first and second fluids are delivered.

7. The method of claim 6, wherein the first infusion pump is configured to delivery a first anesthesia drug, and the second infusion pump is configured to deliver a second anesthesia drug, wherein the method further comprises:
determining a decrease in a flow of the delivery of the second anesthesia drug, and
increasing the flow of the delivery of the first anesthesia drug to maintain the patient in the predetermined hypnotic state of anesthesia based on the decrease in the flow of the delivery of the second anesthesia drug,
wherein the increase in the flow is not provided to the closed-loop control algorithm.

8. The method of claim 1, wherein delivery of the first and second fluids cause an effect, wherein the effect comprises a stable cardiac output, and the sensor measurements comprise a measure of the patient's cardiac output.

9. The method of claim 1, further comprising:
detecting, via a sensor operably connected to an infusion line of the first fluid, a type of the first fluid flowing through the infusion line;
preventing, by an infusion communication system based on intercepting commands sent to the first and second infusion pumps, the first infusion pump from delivering the first fluid or the second infusion pump from delivering the second fluid until the detected type of the first fluid is confirmed as being scheduled for delivery by the first and second infusion pumps.

10. The method of claim 1, further comprising:
identifying an infusion set used for the first fluid; and
preventing, by an infusion communication system based on intercepting commands sent to the first and second infusion pumps, the first infusion pump from delivering the first fluid or the second infusion pump from delivering the second fluid until the infusion set is confirmed as being scheduled for delivery by the first and second infusion pumps.

11. An infusion device control system, comprising:
an infusion device comprising at least one of the first and second infusion pumps;
at least two connection ports configured to communicatively connect to the infusion device and the sensor; and
wherein the infusion device control system is configured to perform a method according to claim 1.

12. A system, comprising:
one or more processors;
a memory device comprising non-transitory computer-readable instructions that, when executed by the one or more processors, cause the one or more processors to perform a method according to claim 1.

13. A memory device comprising non-transitory computer-readable instructions that, when executed by one or more processors, cause the one or more processors to perform a method according to claim 1.

14. A method comprising, by an infusion device control system:
intercepting a first command provided to a first infusion pump by a closed-loop control algorithm, the first command configured to adjust a first therapy provided by the first infusion pump according to sensor measurements provided to the closed-loop control algorithm;
receiving a therapy target control parameter for modifying the first therapy based on a real time parameter of a second therapy provided by a second infusion pump;
receiving, for the first and second infusion pumps, real time operating parameters and infusion status information; and
modifying the intercepted first command based on the real time operating parameters and infusion status information and the therapy target control parameter to control a therapeutic effect of the first and second therapies.

15. The method of claim 14, further comprising:
receiving, based on the infusion status information, an indication of a delivery failure of the first therapy; and
modifying the intercepted first command responsive to the indication of the delivery failure.

16. The method of claim 14, wherein the first therapy comprises a first administration of a first medication to a patient, and the second therapy comprises a second administration of a second medication to the patient, wherein the method further comprises:
receiving an indication that a container for the second medication is nearing empty and that the second infusion pump is operating at a high flow rate; and
causing, responsive to the indication, the second infusion pump to slow a flow rate of the second administration.

17. The method of claim 14, further comprising:
determining, based on the infusion status information of the second infusion pump, that an infusion line of the second infusion pump is occluded,
modifying the intercepted first command to cause a flow rate of the first infusion pump to be adjusted.

18. The method of claim 14, further comprising:
monitoring, by an infusion communication system, commands sent by the closed-loop control algorithm to the first and second infusion pumps;
intercepting, by the infusion communication system based on the monitoring, a second command prior to or contemporaneous with intercepting the first command;
determining that the first command should be processed prior to the second command to control the therapeutic effect; and
delaying the second command from being provided to the second infusion pump until the first command is adjusted and provided to the first infusion pump.

19. The method of claim 14, wherein the therapeutic effect is a predetermined hypnotic state of anesthesia, and the sensor measurements comprise electroencephalographic activity of a patient to which the first and second therapies are provided.

20. The method of claim 19, wherein the first therapy comprises a first administration of a first anesthesia drug, and the second therapy comprises a second administration of a second anesthesia drug, wherein the method further comprises:
determining a decrease in a flow of the second administration, and
increasing the flow of the first administration to maintain the patient in the predetermined hypnotic state of anesthesia based on the decrease in the flow of the second administration,
wherein the increase in the flow is not provided to the closed-loop control algorithm.

21. The method of claim 14, wherein the therapeutic effect comprises a stable cardiac output, and the sensor measurements comprise a measure of the patient's cardiac output.

22. The method of claim 14, further comprising:
detecting, via a sensor operably connected to an infusion line of the first therapy, a type of fluid flowing through the infusion line;
preventing, by an infusion communication system based on intercepting commands sent to the first and second infusion pumps, the first infusion pump from providing the first therapy or the second infusion pump from providing the second therapy until the detected type of fluid is confirmed for the first and second therapy.

23. The method of claim 14, further comprising:
identifying an infusion set used for the first therapy; and
preventing, by an infusion communication system based on intercepting commands sent to the first and second infusion pumps, the first infusion pump from providing the first therapy or the second infusion pump from providing the second therapy until the infusion set is confirmed for the first and second therapy.
